(19) United States
(12) Patent Application Publication
(10) Pub. No.: US 2007/0253994 A1
(43) Pub. Date: Nov. 1, 2007

(54) Intraspinal Drug Delivery Methods and Devices To Alleviate Chronic Pelvic Pain

(75) Inventor: Keith R. Hildebrand, Houlton, MN (US)

Correspondence Address: MEDTRONIC, INC., 710 MEDTRONIC PARKWAY NE, MINNEAPOLIS, MN, 55432-9924, US

(73) Assignee: Medtronic, Inc., Minneapolis, MN (US)

(21) Appl. No.: 11/380,814

(22) Filed: Apr. 28, 2006

Related U.S. Application Data
(60) Provisional application No. 60/746,005, filed on Apr. 28, 2006.

(51) Int. Cl. A61K 38/08; A61K 31/407; A61K 31/192; A61K 31/195

(52) U.S. Cl. 424/422; 514/16; 514/561; 514/411; 514/569; 514/570

(57) ABSTRACT

The disclosure describes methods and systems for alleviating chronic pelvic pain in a subject by intraspinally a therapeutically effective amount of a chronic pelvic pain-alleviating drug. The system includes drug delivery devices and systems for the controlled administration of drugs for alleviation of pelvic pain. The system may deliver drug therapy for pelvic pain in men or women.

### FIELD OF THE INVENTION

This invention relates to drug delivery methods and devices for alleviating chronic pelvic pain in a subject. More specifically the invention relates to methods and systems for administering therapeutic compounds intraspinally to alleviate chronic pelvic pain.

### BACKGROUND OF THE INVENTION

Chronic pelvic pain may occur in both men and women of all ages and results from a variety of injuries and disorders. It is a common and debilitating problem that can significantly impair the quality of life of the patient suffering from it. Chronic pelvic pain occurs in the pelvic or abdominal region and can last for up to six months or longer.

In men, chronic pelvic pain may result from chronic idiopathic prostatitis (also referred to as nonbacterial prostatitis or chronic pelvic pain syndrome), chronic bacterial prostatitis or interstitial cystitis where the symptoms of both typically include in addition to pelvic pain, urinary urgency and frequency, sexual dysfunction and in most cases patients have a nonrelaxing pelvic floor upon physical examination. The most common treatment for these disorders involves pharmacologic treatments typically orally administered such as antibiotics, anti-inflammatory agents, alpha blockers, anti-spasmodics, analgesics, allopurinol, and muscle relaxants. Alpha blockers have successfully treated symptoms of prostatitis, although adverse event rates have been high. Muscle relaxants have shown significant improvement in small studies for patients with sphincter dyssynergia or muscle spasm. Anti-inflammatory agents, such as pentosan polysulfate, have proven successful for approximately 40% of patients with chronic nonbacterial prostatitis.

Other types of chronic pelvic pain experienced by men include chronic testicular pain (CTP), post vasectomy pain, genitofemoral neuralgia and other pain originating from the testicles, groin, or abdomen. The incidence of patients with CTP, also referred to as orchialgia, orchidynia, or chronic scrotal pain, is large and may be caused by on-going inflammation of the testicle (orchitis) or epididymis (epdidymitis), trauma, tumors, hernia, torsion (twisting of the testicle), varicocele, hydrocele, spermatocele polyarteritis nodosa, and previous surgical interventions such as vasectomy and hernia surgery.

Typically, testicle removal and spermatic cord denervation procedures are used to treat CTP. In spermatic cord denervation procedures, nerves in or adjacent to the spermatic cord, i.e., the genitofemoral nerve or sympathetic nerves, are severed or permanently removed. Such procedures may result in permanent and substantial pain relief regardless of the origin of pain. However, severing or removing these nerves may result in loss of sensation in the testicle and/or scrotum, loss of the cremasteric reflex which may cause fertility issues, and even loss of blood flow causing the testicle to die. Therapeutic nerve blocks may also be used to treat CTP, but generally only relieve pain temporarily.

Chronic pelvic pain is also a common medical problem affecting women today. Sources of pain may include injury to nerves resulting from surgical procedures, non-surgical conditions, vulvodynia which can be very debilitating but has no obvious source, and interstitial cystitis (painful bladder syndrome). Surgical procedures that may injure nerves in the pelvic region resulting in pelvic pain may include urological operations in the pelvic area, gynecological surgery, and hysterectomy. Non-surgical conditions which cause pain in women include adhesions, endometriosis, and pelvic congestion.

### SUMMARY OF THE INVENTION

The present invention relates to drug delivery methods and devices for alleviating chronic pelvic pain in a mammalian subject in need of such treatment that includes administering to the subject intraspinally a therapeutically effective amount of a pelvic pain-alleviating drug. Embodiments of the method of the invention include administration of the drug into the epidural, peridural and intrathecal spaces located within the subject's spinal region. In another aspect of the invention, the chronic pelvic pain-alleviating drug is delivered to a target site selected based upon the source of the chronic pelvic pain. In one embodiment, the target site may be one of the epidural, peridural, and intrathecal spaces located below the sacrum and coccys when the source of the pelvic pain is in the lower pelvic region. In another embodiment, the target site may be one of the epidural, peridural, and intrathecal spaces located between spinal segments T10 to S4.

In one aspect of the invention the method comprises using a drug delivery device comprising a pelvic pain-alleviating drug wherein the drug delivery device controls the sustained administration of an amount of the drug effective to alleviate chronic pelvic pain in a controlled manner over a predetermined duration of time. In the practice of the invention, the administration is localized and sustained. The administration may occur over a period of from about at least one day to about six months or from at least one week to about four months or from at least two weeks to about three months. The administration may be continuous, periodic or patterned. A patterned mode of administration comprises releasing a predetermined amount of the chronic pelvic pain-alleviating drug in a predetermined pattern, generally a substantially regular pattern, over a pre-selected period of time (e.g., other than a period associated with, for example a bolus injection) where the duration of the period of time that passes between each release of the drug varies in length in a patterned fashion.

In one embodiment of the invention, the drug delivery device is desirably a depot. In other embodiments, the drug delivery device an external or implanted drug pump system that may include a catheter coupled to the pump. In yet another embodiment of the invention the drug delivery device may be a depot contained with a pump.

Pelvic pain-alleviating drugs useful in the method and device of the invention includes drugs that may be administered to a subject intrathecally, peridurally or epidurally at a dose effective to alleviate chronic pelvic pain as determined by well known means without significant side effects. Such drugs may include gabapentin, sufentanil, bupivacaine, ketorolac, baclofen, ropivacaine, ketamine, octreotide, neostigmine, droperidol, despiramine, and the like.

Various embodiments described in this disclosure may provide one or more advantages over existing methods, devices and systems used to alleviate chronic pelvic pain.

### DETAILED DESCRIPTION OF THE INVENTION

The present invention relates to methods and devices for decreasing, eliminating, or managing chronic pelvic pain by providing direct and controlled administration of a therapeutically effective dose of a pelvic pain-alleviating drug to a patient intrathecally, peridurally, or epidurally.

For the purposes of this disclosure, the term “drug” means any pharmacological or therapeutic agent or combination of agents delivered to provide therapy to a patient (human or non-human animal). The drugs will typically be liquids or materials contained in liquid carriers as either solutions or mixtures (although where used herein, the term “solution” refers to both solutions and mixtures).

As used herein the term “therapeutically effective amount” means an amount of a drug which is effective to achieve a desired therapeutic effect, e.g., alleviation of chronic pelvic pain. The precise desired therapeutic effect (e.g., the degree of pain relief, the cause of the pain relief, etc.) will vary according to the condition to be treated and a variety of other factors that are known by those of ordinary skill in the art.

It will be understood that the amount of chronic pelvic pain-alleviating drug delivered and the target site of the delivery may be altered based upon the response of a patient to the drug. Any measure of pelvic pain improvement or worsening may be used to evaluate whether a therapy modification may be appropriate. Such determinations can be readily made by, for example, a physician attending to the subject's care. In an embodiment, a Visual Analog Scale (VAS) is used to assess pain. The VAS is typically either a horizontal or vertical straight line; usually 10 cm in length with the descriptors of “least possible pain” or “no pain” on one end and “worst possible pain” on the other. The patient marks on the line where their pain level is at the present moment. The distance from the patient's mark to the end of the line is the measure of severity of the pain. The measurement is reproducible, as shown in the correlation coefficients between successive measurements. It is one of the most sensitive measurements of pain. The VAS is easy to administer and understand. It has been administered to children as young as 5 and they were able to use the scale. Alternatively, other quality of life instruments can be used to measure impact (i.e., SF-36).

In an embodiment of the invention, the effective amount to treat chronic pelvic pain will be the amount of drug required to alleviate chronic pelvic pain by an amount of at least about 25% based upon a pain assessment such as VAS and desirably by an amount of at least about 50% without the subject experiencing side effects that significantly diminish the subject's quality of life. Additionally, the amount of a drug may be considered to be a “therapeutically effective amount” for subjects whose chronic pelvic pain is a result of chronic prostatitis, interstitial cystitis or other urinary disorders resulting in increased frequency and urgency to urinate, if, in addition to some alleviation of pain, the frequency and urgency to urinate is decreased. In yet another embodiment, this invention may allow the subject to decrease or eliminate oral medications that may have severe side effects. Examples include narcotics that may limit living a normal life (i.e., cognitive capability may be diminished which may impact driving, intellectual or high motor jobs). Further, it may allow a level of pain relief unachievable via oral or alternative medication or medical procedures.

The amount of the chronic pelvic pain-alleviating drug and target site of its delivery may be adjusted based upon the presentation and severity of side effects in a patient. Side effects may be recognizable by the patient, a physician attending to the care of the patient, other health care professionals, and the like. A physician or other health care professional may adjust therapy parameters based on side effects. Side effects which may be associated with some chronic pelvic pain-alleviating drugs useful with the method of the invention include: dizziness, insomnia, lightheadedness, changes in blood pressure, gastrointestinal disturbances, sexual dysfunction in males, nausea and/or vomiting. Certain classes of drugs that may be useful chronic pelvic pain-alleviating drugs, such as opioid compositions may have particular side effects to consider. Opioid compositions may cause significant respiratory depression and may cause respiratory arrest if given too much too rapidly.

Effective dosages for use in methods as described herein can be determined by those of skill in the art, particularly when effective systemic dosages are known for a particular therapeutic agent. Dosages may typically be decreased by at least 90% of the usual systemic dose if the therapeutic agent is provided in a targeted fashion. In other embodiments, the dosage is at least 75%, at least 80% or at least 85% of the usual system dose for a given condition and patient population. Dosage is usually calculated to deliver a minimum amount of one or more therapeutic agent per day, although daily administration is not required. If more than one pharmaceutical composition is administered, the interaction between the same is considered and the dosages calculated. Intrathecal dosage, for example, can comprise approximately ten percent of the standard oral dosage. Alternatively, an intrathecal dosage is in the range of about 10% to about 25% of the standard oral dosage.

“Target site” as used herein is used to refer to an area of the body to which the drug is administered. Target sites desirably used with the methods of this invention include specific regions within the spinal canal. As used herein, the term “spinal region”includes the spinal canal (including the spinal cord, intrathecal space, dura, epidural space, etc.), vertebrae, spinal discs, nerve roots, and the ligaments, tendons and muscles in between and surrounding the vertebrae. In one embodiment of the invention, the target site is intrathecal. In other embodiments the target site is in the epidural or peridural spaces of the spinal region. The target sites for the administration of a drug to alleviate pelvic pain in subjects also experiencing bladder or pelvic floor disorders is desirably in the epidural, peridural or intrathecal spaces in the spinal region below the sacrum and/or coccys, desirably between Co1 and L1, between S5 and S1, between S2 and L1, or between T10 and S4. In one embodiment, the target site is the spinal region that may be accessed through the sacral hiatus or the sacral foramen.

In one embodiment of this invention, prior to administering a drug delivery device of the invention for a sustained period of time, the subject's physician may wish to evaluate the suitability of one or more target sites. To do so, the physician may deliver a bolus of therapeutically effective amount of a chronic pelvic pain-alleviating drug to one or more predetermined target sites within a patient's spinal region and determine whether the patient experiences any side effects from the drug as well as to determine whether the dosage may be effective for that patient. If the injection of drug at the site ameliorates the patient's pain, a physician may conclude that sustained administration over a period of days, weeks or months may be efficacious, and may proceed to surgically implant or otherwise administer drug delivery devices in accordance with the invention.

As used herein a “controlled administration system” is a direct administration system to deliver chronic pelvic pain-alleviating drugs to a target site within the spinal column of a subject, and includes, but is not limited to, a depot, an osmotic pump, infusion pump, implantable mini-pumps, a peristaltic pump, other drug pumps, or a system administered locally by insertion of a catheter at or near a target site, the catheter being operably connected to a drug delivery pump. It is understood that pumps can be internal or external as appropriate.

In another embodiment, the drug delivery device may be a depot. Suitable depots may take the form of capsules, microspheres, particles, rods, gels, coatings, matrices, wafers, pills, and the like. A depot may comprise a biopolymer. The biopolymer may be a sustained-release biopolymer and may be biodegradable. The depot may be deposited at or near, generally in close proximity, to a target site, such as a spinal or perispinal location. Examples of suitable sustained release biopolymers include but are not limited to poly(alpha-hydroxy acids), poly(lactide-co-glycolide) (PLGA), polylactide (PLA), polyglycolide (PG), polyethylene glycol (PEG) conjugates of poly(alpha-hydroxy acids), polyorthoesters, polyaspirins, polyphosphagenes, collagen, starch, chitosans, gelatin, alginates, dextrans, vinylpyrrolidone, polyvinyl alcohol (PVA), PVA-g-PLGA, PEGT-PBT copolymer (polyactive), methacrylates, poly(N-isopropylacrylamide), PEO-PPO-PEO (pluronics), PEO-PPO-PAA copolymers, PLGA-PEO-PLGA, or combinations thereof. Depots that may be useful to deliver chronic pelvic pain-alleviating drugs of the invention include without limitation those described in Chen et al. U.S. Published Application No. 2005/0079202, U.S. Pat. No. 5,980,927 and in co-pending U.S. application being filed on even date herewith entitled: “SCREENING CANDIDATES FOR IMPLANTABLE INFUSION DEVICES” the teachings of each of the above is incorporated herein by reference in its entirety.

It is desirable that the drug delivery device be able to accurately, precisely and reliably deliver the intended amount of drug over the intended period of time. Many chronic pelvic pain-alleviating drugs are expensive, especially those formulated to retain stability and efficacy over extended periods of time. Thus, the ability to efficiently formulate, process, package and deliver the drugs delivered via the controlled administration system with minimal loss of drug stability and efficacy is desirable. In a preferred embodiment, compositions suitable for controlled administration systems of the instant invention will be carefully formulated for the desired therapeutic effect. The drug itself may be on a continuum of rapid-acting to long-acting or it may be formulated to a continuum of rapid release or sustained release. Still further, the options for delivery of a therapeutically effective amount of a chronic pelvic pain-alleviating drug on a continuum and includes but is not limited to rapid and repeating delivery at intervals ranging to continuous delivery. Delivery may occur at a desired site over a desired period of time for adequate distribution and absorption in the patient.

The controlled administration system of the invention includes, for example, an infusion pump that administers a chronic pelvic pain-alleviating drug through a catheter the proximal end of which is near the predetermined target site, an implantable mini-pump, an implantable controlled release device (such as, for example, the device described in U.S. Pat. No. 6,001,386), and a sustained release delivery system (such as the system described in U.S. Pat. No. 6,007,843).

Potential drug delivery devices suitable for adaptation for the method of the invention include but are not limited those described, for example, in U.S. Pat. No. 6,551,290 (Elsberry, et al.), which describes a medical catheter for target specific drug delivery; U.S. Pat. No. 6,571,125 (Thompson), which describes an implantable medical device for controllably releasing a biologically-active agent; U.S. Pat. No. 6,594,880 (Elsberry), which describes an intraparenchymal infusion catheter system for delivering therapeutic agents to selected sites in an organism; and U.S. Pat. No. 5,752,930 (Rise, et al.), which describes an implantable catheter for infusing equal volumes of agents to spaced sites.

In one embodiment, a drug delivery catheter is inserted into the spinal region at a level below the end of the spinal cord then threaded up to the desired level. Many catheters are very limp, making it difficult or impossible to steer the catheter into a desired specific area of the spinal region. A guidewire may be used to stiffen the catheter during placement. The guidewire is then typically removed before the catheter is used to administer drugs.

In one embodiment, the chronic pelvic pain-alleviating drug is desirably delivered to the spinal region below the sacrum. A depot may be delivered to that space via a drug delivery catheter. The techniques for such delivery method are well known in the art. In one embodiment, the Seldinger technique is used and an introducer having a lumen is used to enter the spinal space through one of the sacral hiatus or sacral foramens, a guidewire is passed through the introducer, the introducer is removed and the catheter is advanced over the wire until it is in position for drug delivery.

One method of evaluating a particular drug's usefulness as a chronic pelvic pain-allevating drug in the methods of the invention, particularly, for chronic pelvic pain associated with chronic idiopathic prostates and interstitial cystitis is to examine the effect of potential candidates on the micturition reflex of female rats in the interstitial cystitis (IC) model of visceral pain. The results obtained with this animal model will be only one factor used in evaluating the efficacy of a particular drug for alleviating chronic pelvic pain with the method of the invention as animals cannot assess whether administration of a particular drug alleviates pelvic pain associated with such disorders.

We have used the above-mentioned model to evaluate seven drugs that have been used to treat pain in humans and one vehicle control. Baseline values were obtained in response to saline infusion immediately prior to testing. Acetic acid infusion was then used to sensitize the bladder. Acetic acid infusion significantly increased the frequency of the micturition reflex, but had no effect on micturition amplitude. Test compounds were administered intrathecally in two ascending doses on each animal. Bupivacaine, sufentanil, gabapentin, baclofen, desipramine, clonidine and ketorolac were tested as follows.

Rats (female) weighing 200 to 250 grams were anesthetized with urethane (1.2 g/kg), to achieve a stable level of anesthesia lasting for the entire experiment. The bladder was catheterized transurethrally with a polyethylene catheter (PE-10), which was secured in place by taping it to the tail of the rat. The catheter was attached to a pressure transducer for continuous measurement of intravesicle pressure. The bladder was infused by a syringe pump at a rate of 0.1 mL/min. The signal was amplified (gain+10,000), filtered and digitized. The endpoint of the experiment was bladder function as assessed by cystometrograms, which measure bladder pressure as a function of time. Parameters analyzed were micturition frequency, amplitude, interval and maximum micturition pressure. Pressure was measured in mm Hg. Empirical data analysis indicates that micturition reflex frequency is, specifically, the most reliable response parameter for evaluation of drug effects. Baseline cystometrograms were recorded during 30-50 minutes of continuous salin infusion at a rate of 0.1 mL/min. Then the bladder was infused with acetic acid (0.5%) at the same infusion rate. Cystometrograms were obtained during one hour of pre-drug infusion (acetic acid baseline) and after two intrathecal drug doses lasting up to 60 minutes each. Acetic acid infusion continued during drug administration.

All drugs tested were administered by intrathecal injection. Dosage volume was 10 μl except for the higher doses of sufentanil and ketorolac, which were delivered in a volume of 20 μl. Table 1 summarizes the study design.

TABLE 1

| | | | | | | Group |
|---|---|---|---|---|---|---|
| Group | Treatment | Test Compound | Doses | | Route | Size (n) |
| 1 | Acetic acid | Bupivacaine HCl | 2, 10 | μg | intrathecal | 10 |
| 2 | Acetic acid | Sufentanil citrate | 0.2, 1.0 | μg | intrathecal | 10 |
| 3 | Acetic acid | Clonidine HCl | 2, 10 | μg | intrathecal | 10 |
| 4 | Acetic acid | Baclofen | 0.2, 2.0 | μg | intrathecal | 10 |
| 5 | Acetic acid | Gabapentin | 20, 100 | μg | intrathecal | 10 |
| 6 | Acetic acid | Desipramine HCl | 20, 100 | μg | intrathecal | 10 |
| 7 | Acetic acid | Ketorolac tris | 100, 500 | μg | intrathecal | 10 |
| 8 | Acetic acid | Vehicle (saline) | 10 | μl | intrathecal | 10 |

Bupivacaine, sufentanil, gabapentin, baclofen, desipramine, and ketorolac suppressed micturition reflex frequency in a dose-dependent fashion. Clonidine exerted no measurable effect at the doses tested. Sufentanil is an opioid and may cause respiratory depression and respiratory arrest if given too much too rapidly. Respiratory distress observed in some rats at the higher dose of 1.0 μg.

Gabapentin and desipramine had statistically very similar effects. Both were effective at reducing the reflex frequency at the higher dose but not at the lower dose. Each of these drugs at the higher dose reduced the frequency of acetic-acid-induced bladder contractions to a level comparable to the normal frequency of contraction seen with saline infusion. Neither of these drugs suppressed the amplitude of bladder contractions suggesting that the motor function of the bladder was not inhibited. Bupivacaine appeared to suppress the micturition frequency at both doses but had no effect on the amplitude of contraction. At both doses, bupivacaine reduced the frequency of acetic-acid-induced contractions below the rate observed during saline infusion. Sufentanil produced dose-dependent reductions in the freqeuncy of acetic-acid-induced contractions to a level below that observed during saline infusion. At 1 μg, sufentanil significantly suppressed the amplitude of the bladder contractions. It was the only drug tested that produced this effect. Baclofen and Ketorolac each reduced the acetic-acid-induced contraction frequency in a dose-dependent manner without suppressing it below the normal rate of contraction observed with saline infusion. For Baclofen this was statistically significant only at the higher dose. Ketorolac results were the most consistent of any group. In each of the ten rats tested, the high dose reduced the reflex frequency. For the lower dose, 8 of 10 rats exhibited a lower reflex frequency. Neither baclofen nor ketorolac reduced the amplitude of bladder contraction. Injection of the saline vehicle had no effect on micturition frequency.

The preceding specific embodiments are illustrative of the practice of the invention. It is to be understood, therefore, that other expedients known to those skilled in the art or disclosed herein may be employed without departing from the invention or the scope of the appended claims.

What is claimed is:

1. A method for alleviating chronic pelvic pain in a mammalian subject comprising delivering a depot comprising a therapeutically effective amount of a chronic pelvic pain-alleviating drug intraspinally to a target site in the subject and administering the drug to the subject.

2. The method of claim 1, wherein the depot is delivered to the subject's spinal region via an epidural, peridural or intrathecal route.

3. The method of claim 1, wherein the drug administration is localized and sustained.

4. The method of claim 1, wherein the pelvic pain includes at least one of chronic groin pain, chronic testicular pain (CTP), post vasectomy pain, ilioinguinal neuralgia, chronic idiopathic prostatitis, chronic bacterial prostatitis, vulvodynia, and interstitial cystitis.

5. The method of claim 1, wherein the drug comprises at least one of gabapentin, sufentanil, bupivacaine, ketorolac, baclofen, ropivacaine, ketamine, octreotide, neostigmine, desipramine, and droperidol.

6. The method of claim 1, wherein the drug administration occurs over a period of from about at least one day to about three months.

7. The method of claim 1, wherein the drug administration is continuous.

8. The method of claim 1, wherein the drug administration is periodic.

9. The method of claim 1, wherein the drug has a controlled release rate.

10. The method of claim 9, wherein the controlled release rate is from about 24 hours to about 31 days.

11. The method of claim 9, wherein the controlled release rate is from about at least one day to about three months.

12. The method of claim 1, wherein the depot is delivered via a drug delivery device.

13. The method of claim 12, wherein the drug delivery device is implanted in a subject at or near a target site.

14. The method of claim 12, wherein the drug delivery device comprises a drug delivery catheter having a proximal end and a distal end, the proximal end having an opening to deliver a drug in situ and the therapeutically effective amount of the stress incontinence-reducing drug is administered via the catheter.

15. The method of claim 1, wherein the target site is within the subject's spinal region below the coccys or sacrum between Co1 and L1.

16. The system of claim 1, wherein the target site is within the subject's spinal region between S2 and L1.

17. The system of claim 1, wherein the target site is within the subject's spinal region between T 10 and S4.

18. The method of claim 1, wherein the target site is the spinal region accessible through the sacral hiatus or one of the sacral foramens.

19. A method for alleviating chronic pelvic pain in a subject comprising administering a therapeutically effective amount of a chronic pelvic pain-alleviating drug epidurally, peridurally or intrathecally, wherein the therapeutically effective amount and type of chronic pelvic pain-alleviating drug is determined to diminish at least one adverse side effect of the drug.

20. The method of claim 19 wherein said adverse side effect is dizziness, insomnia, respiratory depression, lightheadedness, changes in blood pressure, gastrointestinal disturbances, sexual dysfunction, nausea and/or vomiting, urinary retention, decreased cognitive ability, drug dependency, and depression.

21. A device for administering a chronic pelvic pain alleviating-drug to a subject at a sustained rate over a period of time, the device being shaped, sized and adapted for delivering the device epidurally or intrathecally into the spinal region of the subject, the device comprising a biostable polymeric body, wherein the chronic pelvic pain alleviating-drug is loaded into the body or dissolved or dispersed in the polymer in an amount sufficient so that a therapeutically effective amount of the drug will be delivered for a predetermined duration of time after implant to the region of the spine where the device was delivered.

22. The device of claim 21, wherein the predetermined duration of time is from about 24 hours to about 31 days.

23. The device of claim 21, wherein the predetermined duration of time is about 1 week to about three months.

24. The device of claim 21, wherein the predetermined duration of time is about 2 weeks to about six months.

25. The device of claim 21, wherein the chronic pelvic pain-alleviating drug is selected from the group consisting of gabapentin, sufentanil, bupivacaine, ketorolac, baclofen, ropivacaine, ketamine, octreotide, neostigmine, desipramine, and droperidol.

26. A system for administering a chronic pelvic pain-alleviating drug intraspinally in a subject, the system comprising a controlled administration system for providing controlled delivery of a therapeutically effective amount of the chronic pelvic pain-alleviating drug to a target site in a subject in need thereof.

27. The system of claim 26, wherein the controlled administration system comprises a catheter having a proximal end and a distal end, the proximal end having an opening to deliver a drug in situ and a depot comprising the chronic pelvic pain-alleviating drug wherein upon delivery of the depot the target site the release of the drug is controlled.

28. The system of claim 27, wherein the proximal end of the catheter delivers the chronic pelvic pain-alleviating drug within about 1 mm to about 10 cm of the target site.

29. The system of claim 27, wherein the proximal end of the catheter delivers the chronic pelvic pain-alleviating drug within a range of about 1 cm to about 5 cm of the target site.
